(19) United States
(12) Patent Application Publication
(10) Pub. No.: US 2019/0133590 A1
(43) Pub. Date: May 9, 2019

(54) LIGATION CLIP WITH CONTROLLED TISSUE COMPRESSION

(71) Applicant: Covidien LP, Mansfield, MA (US)

(72) Inventor: Paul D. Richard, Shelton, CT (US)

(21) Appl. No.: 16/120,648

(22) Filed: Sep. 4, 2018

Related U.S. Application Data
(60) Provisional application No. 62/581,151, filed on Nov. 3, 2017.

(51) Int. Cl. A61B 17/122

(52) U.S. Cl. CPC A61B 17/122

(57) ABSTRACT

A surgical ligation clip includes a first jaw, a second jaw, and a central beam that is positioned between the first and second jaws. The ligation clip is configured to clamp tissue between the first jaw and the central beam. The central beam is resilient and applies a controlled pressure to a body vessel during clamping of the body vessel to prevent over compression of the body vessel while allowing for proper vessel sealing and minimizing the likelihood of ischemic tissue damage.

### CROSS-REFERENCE TO RELATED APPLICATION

This application claims the benefit of and priority to U.S. Provisional Patent Application No. 62/581,151 filed Nov. 3, 2017, the entire disclosure of which is incorporated by reference herein.

### BACKGROUND

### 1. Technical Description

The present disclosure is directed to ligation clips for sealing body vessels and, more particularly, to ligation clips for sealing body vessels that include structure to apply a controlled pressure to the body vessel.

### 2. Background of Related Art

Ligation clips for sealing body vessels are well known in the surgical arts and are commonly used during a variety of surgical procedures. Typically, ligation clips include first and second jaws that have first ends that are connected to each other by a hinge and have second ends that are pivotable about the hinge in relation to each other between open and clamped positions. Each of the first and second jaws includes a clamping surface. When the ligation clip is positioned about a body vessel, the body vessel is clamped between the clamping surfaces of the first and second jaws. If the body tissue is compressed between the first and second jaws with a clamping force that is too great, ischemic tissue damage may result in the body tissue.

In some surgical procedures, the ligation clip may have to be removed from the body vessel to allow a surgeon to reposition the clip. Some known clips include a latching mechanism to secure the first and second jaws of the ligation clip in the clamped position about the body vessel. In some known clips, the first and second jaws of the ligation clip must be further compressed from the clamped position to release the latching mechanism to move the ligation clip from the clamped position to the open position. This may result in further ischemic tissue damage.

A continuing need exists in the art for a ligation clip that can apply a controlled pressure to a body vessel and can be removed from the body vessel while minimizing damage to tissue of the body vessel.

### SUMMARY

A ligation clip includes a first jaw, a second jaw, and a central beam. The first jaw has a proximal portion, a distal portion, and a clamping surface. The second jaw has a proximal portion, a distal portion, and an inner surface that faces the clamping surface of the first jaw. The proximal portion of the first jaw is connected to the proximal portion of the second jaw. The first jaw is pivotable in relation to the second jaw between open and clamped positions. The central beam extends from the distal portion of the second jaw towards the clamping surface of the first jaw between the clamping surface of the first jaw and the inner surface of the second jaw. The central beam is formed of a resilient material and is spaced from the clamping surface of the first jaw and the inner surface of the second jaw in the open position of the first and the second jaws.

In embodiments, the central beam is integrally formed with the second jaw.

In some embodiments, the central beam has a proximal end that is positioned adjacent to the clamping surface of the first jaw when the ligation clip is in the open position.

In certain embodiments, the central beam has a proximal end that is dimensioned to engage the clamping surface of the first jaw such that the proximal end of the central beam is deformed downwardly towards the inner surface of the second jaw.

In embodiments, the proximal end of the central beam includes a forked structure including first and second spaced tines and the first jaw includes opposite sides. Each of the first and second tines is positioned on one of the opposite sides of the first jaw.

In some embodiments, the proximal portion of each of the first and second jaws includes a base portion and the spaced tines are positioned to engage the base portion as the first and second jaws are moved towards the clamped position.

In certain embodiments, the base portion is rectangular.

In embodiments, the central beam has a thickness and a length, and the thickness decreases along at least a portion of the length from a distal end of the central beam to a proximal end of the central beam.

In some embodiments, the first and second jaws have a length and a thickness, and the thickness decreases along at least a portion of the length of the first and second jaws from a proximal end of the first and second jaws to a distal end of the first and second jaws.

In certain embodiments, the ligation clip is formed from a biocompatible polymeric material that is selected from the group consisting of acetal polyoxymethylene (POM), polyethylene terephthalate (PET), polybutylene terephthalate (PBT), polyoxymethylene, polyetheretherketone (PEEK), polypropylene, and polyethylene.

In some embodiments, the central beam is positioned and configured to be spaced from the first and second jaws in the clamped position.

In certain embodiments, each of the first and second jaws includes a pair of bosses and each of the pair of bosses is configured to engage an applicator to apply the ligation clip to tissue.

In embodiments, each of the pair of bosses projects outwardly beyond an outer surface of the first and second jaws.

In some embodiments, the first jaw includes a first locking element and the second jaw member includes a second locking element wherein the first locking element is movable into engagement with the second locking element to retain the first and second jaws in the clamped position.

In certain embodiments, a proximal end of the base portion of the first jaw is secured to the proximal end of the base portion of the second jaw by a living hinge.

In embodiments, the base portions of the first and second jaws are retained in juxtaposed relation to each other by interlocking structure.

In embodiments, the interlocking structure includes protrusions formed on one of the first and second jaws and recesses defined in the other of the first and second jaws.

In some embodiments, the distal portion of the first jaw includes a first locking element and the distal portion of the second jaw includes a second locking element, wherein the first locking element is configured to engage the second locking element to retain the first and second jaws in the clamped position.

### BRIEF DESCRIPTION OF THE DRAWINGS

Various exemplary embodiments of the presently disclosed ligation clip are described herein below with reference to the drawings, wherein:

FIG. 1 is a side perspective view of an exemplary embodiment of the presently disclosed ligation clip in an open position;

FIG. 2 is a perspective view from a proximal end of the ligation clip shown in FIG. 1 in an open position;

FIG. 3 is a side view of the ligation clip shown in FIG. 1 in an open position;

FIG. 4 is a cross-sectional view taken along section line 4-4 of FIG. 1 with a body vessel positioned between jaws of the ligation clip and the ligation clip in the open position;

FIG. 5 is a cross-sectional view taken along section line 4-4 of FIG. 1 with the body vessel positioned between jaws of the ligation clip as the ligation clip is moved from the open position towards the closed position;

FIG. 6 is a cross-sectional view of the ligation clip shown in FIG. 4 with the body vessel positioned between jaws of the ligation clip as the ligation clip is moved further towards the closed position;

FIG. 7 is a cross-sectional view of the ligation clip shown in FIG. 4 with the body vessel positioned between jaws of the ligation clip and the ligation clip latched in the closed position;

FIG. 8 is a cross-sectional view of the ligation clip shown in FIG. 7 with the body vessel positioned between the jaws of the ligation clip as the ligation clip is compressed to unlatch the ligation clip;

FIG. 9 is a is a side perspective view of another exemplary embodiment of the presently disclosed ligation clip in an open position;

FIG. 10 is a perspective view from a proximal end of the ligation clip shown in FIG. 10 in an open position;

FIG. 11 is a side view of the ligation clip shown in FIG. 10 in an open position;

FIG. 12 is a perspective view from a distal end of the ligation clip shown in FIG. 10 in an open position;

FIG. 13 is a side view of the ligation clip shown in FIG. 10 in the clamped position;

FIG. 14 is a side view of a plurality of ligation clips supported on a thin metal beam after removal of the clips from a mold;

FIG. 15 is a side perspective view of the plurality of ligation clips shown in FIG. 14 supported on a beam;

FIG. 16 is a perspective view from the distal end of another exemplary embodiment of the presently disclosed ligation clip in the open position with a forked structure of a central beam engaged with a first jaw of the ligation clip;

FIG. 17 is a perspective view from the proximal end of the ligation clip shown in FIG. 16 in the open position;

FIG. 18 is a side view of the ligation clip shown in FIG. 17 in the open position with base portions of the first and second jaws disengaged from each other and the forked structure of the central beam disengaged from the first jaw;

FIG. 19 is a side view of the ligation clip shown in FIG. 18 as the jaws of the ligation clip are moved from the open position towards the clamped position with the base portions of the first and second jaws engaged to each other and the forked structure of the central beam portion engaged with the first jaw of the ligation clip;

FIG. 20 is a side view of the ligation clip shown in FIG. 19 in the clamped position with the central beam portion engaged with the first jaw of the ligation clip.

### DETAILED DESCRIPTION OF EMBODIMENTS

The presently disclosed ligation clip will now be described in detail with reference to the drawings in which like reference numerals designate identical or corresponding elements in each of the several views. However, it is to be understood that the disclosed embodiments are merely exemplary of the disclosure and may be embodied in various forms. Well-known functions or constructions are not described in detail to avoid obscuring the present disclosure in unnecessary detail. Therefore, specific structural and functional details disclosed herein are not to be interpreted as limiting, but merely as a basis for the claims and as a representative basis for teaching one skilled in the art to variously employ the present disclosure in virtually any appropriately detailed structure.

In this description, the term “proximal” is used generally to refer to that portion of the device that is closer to a clinician, while the term “distal” is used generally to refer to that portion of the device that is farther from the clinician. In addition, the term “clinician” is used generally to refer to medical personnel including doctors, nurses, and support personnel.

The presently disclosed ligation clip includes a first jaw, a second jaw, and a central beam that is positioned between the first and second jaws. The ligation clip is configured to clamp tissue between the first jaw and the central beam. The central beam is resilient and applies a controlled pressure to a body vessel during clamping of the body vessel to prevent over compression of the body vessel while allowing for proper vessel sealing and minimizing the likelihood of ischemic tissue damage. When the ligation clip is in a clamped configuration about the body vessel, the central beam is spaced from an inner surface of the second jaw. This spacing allows the ligation clip to be unlatched and removed from the body vessel without over compressing the body vessel.

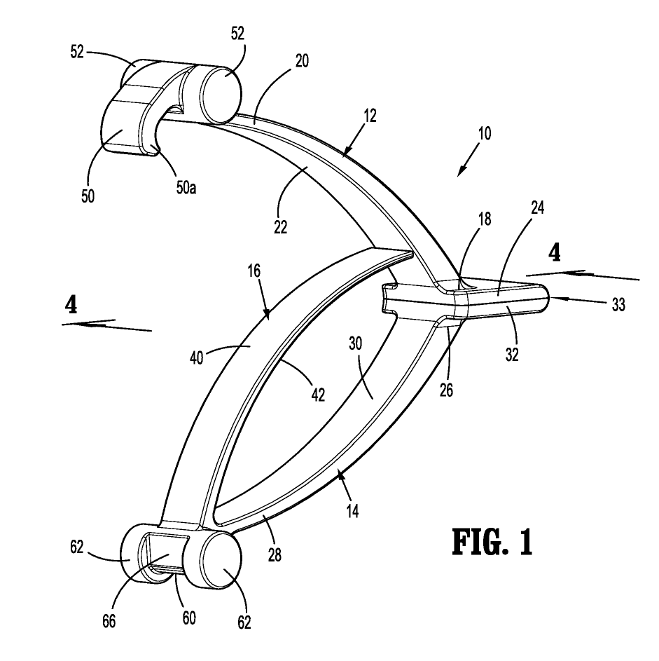

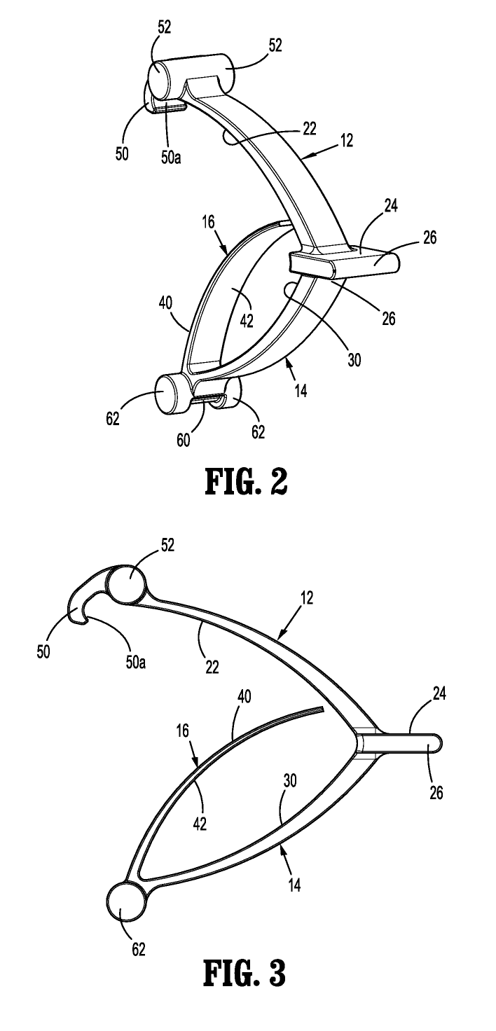

Referring to FIGS. 1-3, an exemplary embodiment of the presently disclosed ligation clip is shown generally as 10 and includes a first jaw 12, a second jaw 14, and a central beam 16. The first jaw 12 includes a proximal portion 18, a distal portion 20, and a clamping surface 22. In embodiments, the proximal portion 18 of the first jaw 12 includes a base portion 24 that is described in further detail below. The second jaw 14 includes a proximal portion 26, a distal portion 28, and an inner surface 30. In embodiments, the proximal portion 26 of the second jaw 14 includes a base portion 32 that is engaged with the base portion 24 of the first jaw 12 to secure the clamping surface 22 of the first jaw 12 in opposition to the inner surface 30 of the second jaw 14. In some embodiments, the first jaw 12 and the second jaw 14 are integrally and/or monolithically formed with each other. In such an embodiment, the base portions 24, 32 are formed as a single piece. Alternately, the first jaw 12 can be formed separately from the second jaw 14 and pivotally secured to the second jaw 14 by connecting the base portions 24, 32 of the first and second jaws 12, 14, respectively, to each other.

The first and second jaws have a length and a thickness. In embodiments, the thickness of the first and second jaws 12, 14 may decrease along all or a portion of their lengths from a proximal end of the first and/or second jaws 12, 14 to a distal end of the first and/or second jaws 12, 14.

In embodiments, the base portions 24, 32 of the first and second jaws 12, 14, respectively, can have a rectangular configuration. The proximal portion 18 of the first jaw 12 can be positioned in abutting relation to the proximal portion 26 of the second jaw 14 and secured together using any known fastening technique, e.g., welding, to define the base 33 of the ligation clip 10. The base 33 may be configured to movably retain the ligation clips 10 within a clip applying apparatus to facilitate sequential application of the clips 10 to a body vessel. For example, the rectangular base 33 can be configured to slide along a slot form in a clip applicator (not shown).

In embodiments, the central beam 16 of the ligation clip 10 extends from the distal portion 28 of the second jaw 14 towards the proximal portion 18 of the first jaw 12 to a position adjacent the clamping surface 22 of the first jaw 12. In embodiments, the central beam 16 is curved and includes a first side 40 defining a convexity that faces the clamping surface 22 of the first jaw 12 and a second side 42 that defines a concavity that faces the inner surface 30 of the second jaw 14. The central beam 16 can be integrally and/or monolithically formed with the second jaw 14. Alternately, the central beam 16 can be formed separately from the second jaw 14 and resiliently mounted to the second jaw 14 using a pivot member (not shown) and a biasing member (not shown). In embodiments, the thickness of the central beam 16 may decrease along all or a portion of its length from a distal end of the central beam to a proximal end of the central beam.

The distal portion 20 of first jaw 12 includes a first locking element 50 and spaced bosses 52. The first locking element 50 includes a curved hooked portion 50a that extends downwardly and proximally to define a portion of a latching mechanism 54 (FIG. 7) that will be described in detail below. The bosses 52 are positioned and configured to engage the jaws of an applicator (not shown) to facilitate application of the ligation clip 10 to tissue.

The distal portion 28 of the second jaw 14 includes a second locking element 60 and a pair of bosses 62. The second locking element 60 defines a protrusion 60a and a recess 60b. The recess 60b of the second locking element 60 is configured to receive the first locking element 50 such that the first locking element releasably engages the curved hook portion 50a of the first jaw 12. The bosses 62 are similar to the bosses 52 of the first jaw 12 and are configured to engage jaws of an applicator (not shown) to facilitate application of the ligation clip 10 to tissue.

A distal wall of the locking element 60 defines a cam surface 66 that is positioned between the bosses 62. The cam surface 66 is curved or angled downwardly and outwardly in a distal direction. The configuration of the cam surface 66 causes the first locking element 50 to flex outwardly in the distal direction as the first and second jaws 12, 14 are moved to the clamped position (FIG. 7) such that as the first locking element 50 passes off of the cam surface 66 the first locking element 50 and snaps into the recess 60b into engagement with the second locking element 60.

In embodiments, the surgical ligation clip 10 may be comprised of a resilient bioabsorbable polymeric material. Examples of suitable bioabsorbable polymers include acetal polyoxymethylene (POM), polyethylene terephthalate (PET), polybutylene terephthalate (PBT), polyoxymethylene, polyetheretherketone (PEEK), polypropylene, and polyethylene or other thermoplastic materials having similar properties that can be injection-molded. The clip may also be comprised of polymer material in combination with radiolucent metal alloys. Alternately, other materials may be used to form the clip 10 including biocompatible metals, plastics and composites.

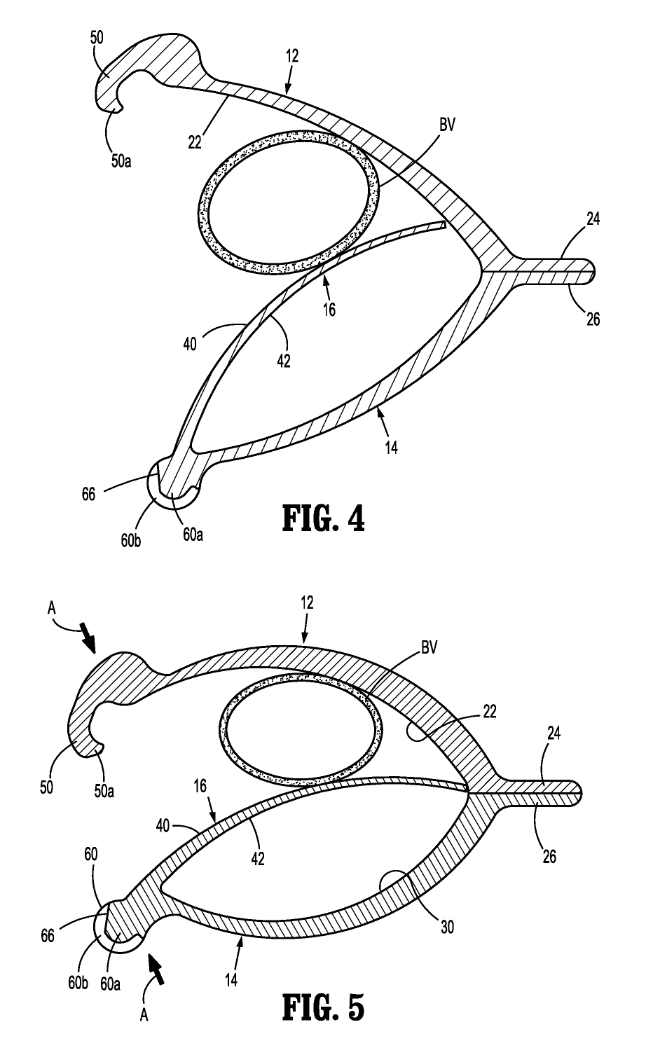

Referring to FIG. 4, when the ligation clip 10 is used to clamp a body vessel “BV”, the ligation clip 10 is placed in the open position about the body vessel “BV” such that the body vessel “BV” is positioned between the first convex side 40 of the central beam 16 and the clamping surface 22 of the first jaw 12. In the open position, the central beam 16 is spaced from both the clamping surface 22 of the first jaw 12 and the inner surface 30 of the second jaw 14.

Referring to FIG. 5, when the first and second jaws 12, 14 are clamped about the body vessel “BV” and moved in the direction indicated by the arrows “A”, the body vessel “BV” begins to be clamped between the clamping surface 22 of the first jaw 12 and the first convex side 40 of the central beam 16. The central beam 16 is resilient and applies a controlled pressure to the body vessel “BV” to prevent over compression of tissue of the body vessel “BV” to allow for proper tissue sealing and to minimize the likelihood of ischemic tissue damage.

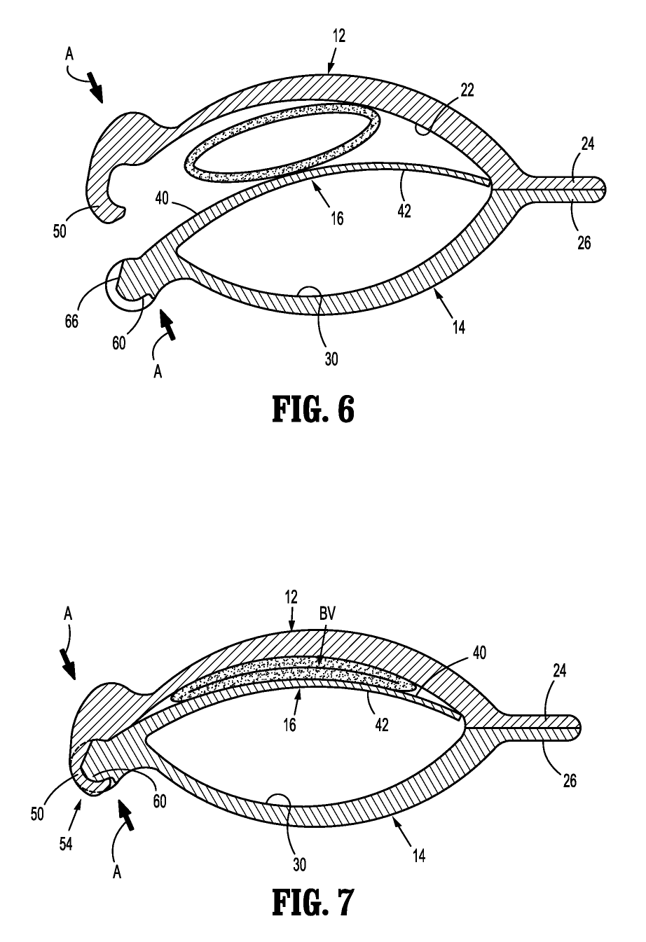

Referring to FIGS. 6 and 7, as the first and second jaws 12, 14 continue to be moved in the direction “A” from the open position towards the clamped position, the first locking element 50 moves into the recess 60b onto the cam surface 66 and eventually passes off of the cam surface 66 such that the first locking element 50 snaps into engagement with the protrusion 60b of the second locking element 60. Once again, the resilience of the central beam 16 applies a controlled pressure to the body vessel “BV” to seal the body vessel “BV” between the central beam 16 and the clamping surface 22 of the first jaw 12.

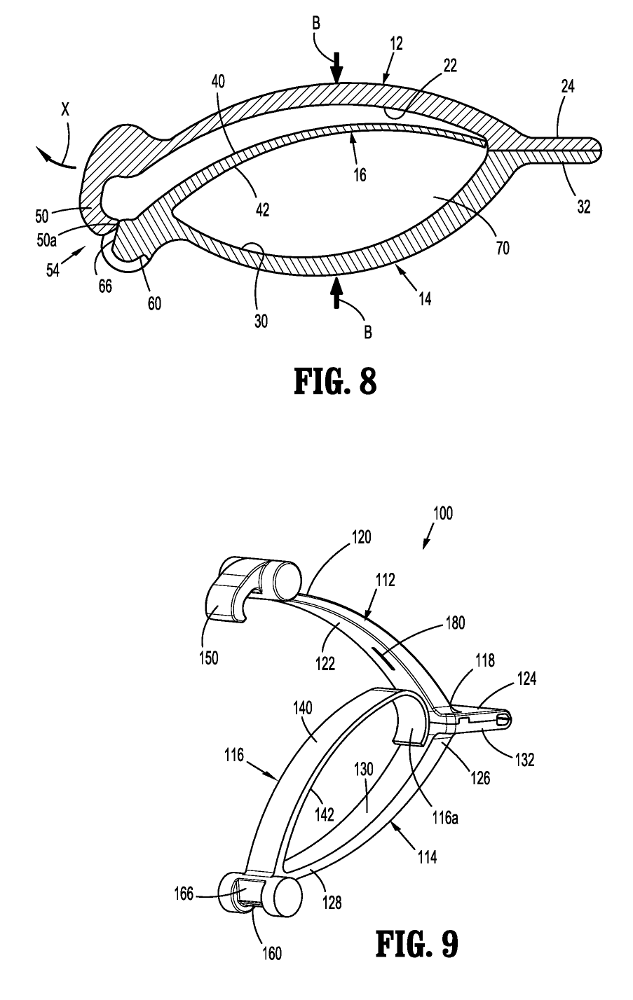

Referring to FIG. 8, in order to disengage the latching mechanism 54 to move the ligation clip 10 from the clamped position to the open position and facilitate removal of the ligation clip 10 from the body vessel “BV”, pressure can be applied to the central portion of the first and second jaws 12, 14 in the direction indicated by arrows “B” using, e.g., a grasper. When pressure is applied to the central portion of the first and second jaws 12, 14, the first locking element 50 deflects outwardly from the second locking element 60 and is disengaged from the second locking element 60 to allow the first and second jaws 12, 14 to move to the open position. Because the central beam 16 is able to flex into a cavity 70 defined between the concave surface 42 of the central beam 16 and the inner surface 30 of the second jaw 14 when the jaws 12, 14 are pressed inwardly in the direction indicated by arrow “B”, over compression of the tissue of the body vessel “BV” is prevented to reduce the likelihood that ischemic tissue damage will occur to tissue during opening of the ligation clip 10.

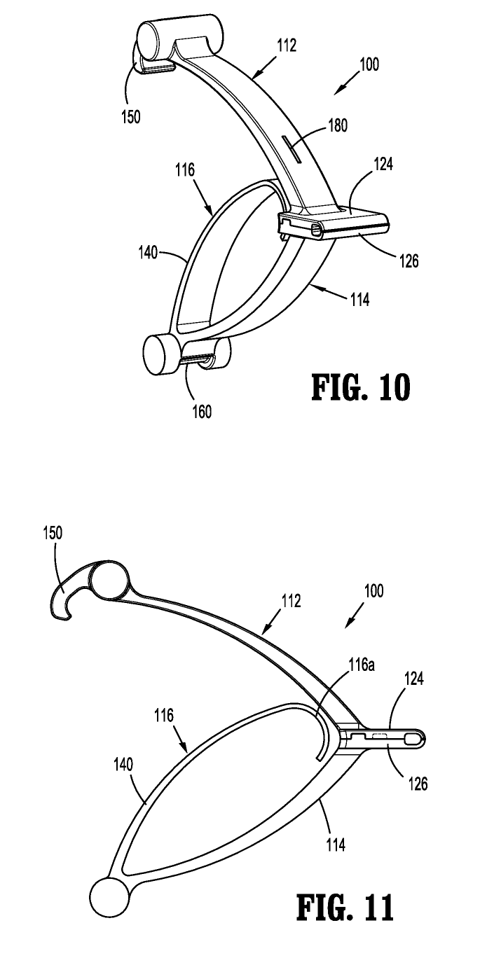

FIGS. 9-13 illustrate another exemplary embodiment of the presently disclosed ligation clip which is shown generally as ligation clip 100. Referring to FIGS. 9-11, the ligation clip 100 is substantially similar to ligation clip 10. Accordingly, only the differences between the ligation clip 100 and the ligation clip 10 will be described in detail. The ligation clip 100 includes a first jaw 112, a second jaw 114, and a central beam 116. The first jaw 112 includes a proximal end 118, a distal portion 120, and a clamping surface 122. In embodiments, the proximal end 118 of the first jaw 112 includes a base portion 124. The second jaw 114 includes a proximal portion 126, a distal portion 128, and an inner surface 130. The proximal portion 126 of the second jaw 114 includes base portion 132 that is engaged with the base portion 124 of the first jaw 112 to secure the clamping surface 122 of the first jaw 112 in opposition to the inner surface 130 of the second jaw 114.

The central beam 116 of the ligation clip 100 extends from the distal portion 128 of the second jaw 114 towards the proximal portion 118 of the first jaw 112 into engagement with the clamping surface 122 of the first jaw 112. In embodiments, the proximal end 116a of the central beam 116 is deformed downwardly towards the inner surface 130 of the second jaw 114. As described above, the central beam 116 may be curved and include a first side 140 defining a convexity that faces the clamping surface 122 of the first jaw 112 and a second side 142 that defines a concavity that faces the inner surface 130 of the second jaw 114. In embodiments, the central beam 116 is integrally and/or monolithically formed with the second jaw 114. Alternately, the central beam 116 can be formed separately from the second jaw 114 and resiliently mounted to the second jaw 114 using a pivot member (not shown) and a biasing member (not shown).

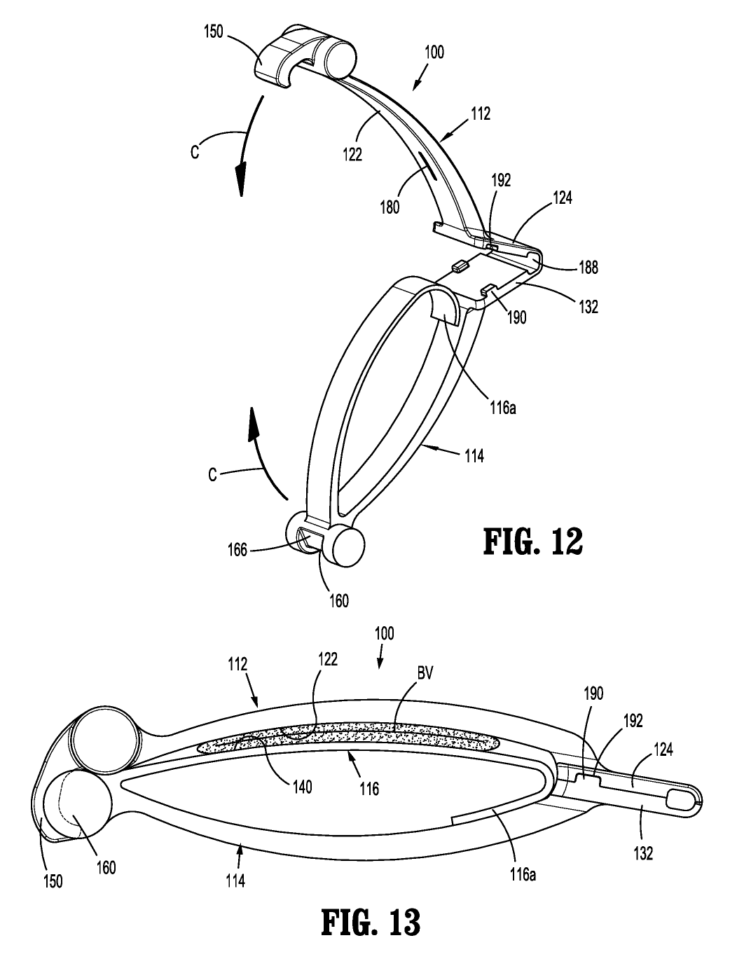

Referring also to FIGS. 12 and 13, in contrast to the base portions 24, 32 of the ligation clip 10 (FIG. 1), the base portions 124, 132 of the first and second jaws 112, 114, respectively, of the ligation clip 100 are attached at their proximal ends by a living hinge 188 (FIG. 12). In addition, each of the base portions 124, 132 of the first and second jaws 112, 114, respectively, includes interlocking structure to secure the base portions 124, 132 together in closed, juxtaposed relation. In embodiments, the interlocking structure includes protrusions 190 formed on one the base portions 124, 132, e.g., base portion 132, and recesses 192 defined in the other of the base portions 124, 132, e.g., base portion 124 (FIG. 12). The protrusions 190 are received within the recesses 192 when the base portions 124, 132 are pivoted towards each other to secure the base portion 124, 132 in juxtaposed relation to each other. It is envisioned that other interlocking or retaining structures may be used to secure the base portions 124, 132 to each other in the closed, juxtaposed relation.

Referring to FIGS. 12 and 13, the ligation clip 100 is applied to a body vessel “BV” in the same manner as ligation clip 10 is applied to a body vessel “BV”. More specifically, when the ligation clip 100 is used to clamp a body vessel “BV” (FIG. 13), the ligation clip 100 is placed in the open position about the body vessel “BV” with the body vessel “BV” positioned between the first convex side 140 of the central beam 116 and the clamping surface 122 of the first jaw 112. In the open position, the flexible beam 116 is spaced from both the clamping surface 122 of the first jaw 112 and the inner surface 130 of the second jaw 114. The first and second jaws 112, 114 are clamped about the body vessel “BV” by moving the first and second jaws 112, 114 in the direction indicated by the arrows “C” in FIG. 12. The central beam 116 is resilient and applies a controlled pressure to the body vessel “BV” to allow proper tissue sealing and to minimize the likelihood of ischemic tissue damage. As the first and second jaws 112, 114 continue to move in the direction of arrows “C” towards the clamped position, the first locking element 150 moves along the cam surface 166 and eventually passes off of the cam surface 166 such that the first locking element 150 snaps into engagement with the second locking element 160. Once again, the resilience of the central beam 116 applies a controlled pressure to the body vessel “BV” to seal the body vessel “BV” between the central beam 116 and the clamping surface 122 of the first jaw 12.

The ligation clip 100 can be released from the body vessel “BV” in the same manner that the ligation clip 10 is released from the body vessel “BV”.

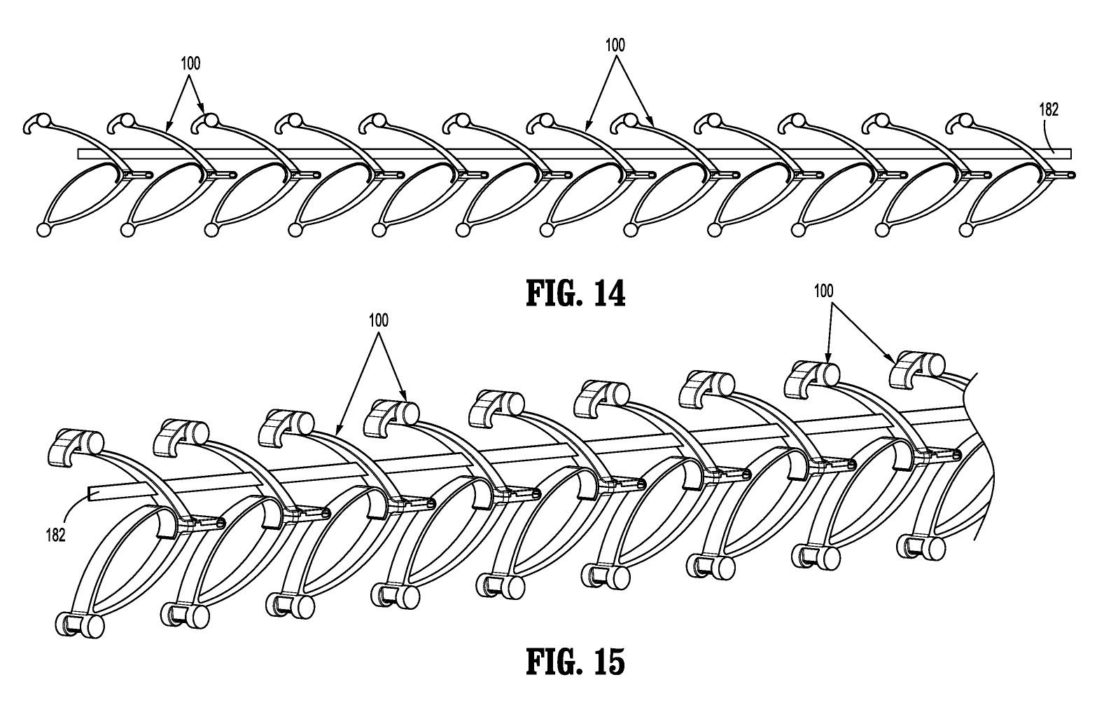

Referring also to FIGS. 14 and 15, in embodiments, the first jaw 112 defines a slot 180 that extends through the first jaw 112. The slot 180 is defined by a beam 182 (FIG. 14) during a molding process such that a plurality of ligation clips 100 are supported along the beam 182 after the molding process is completed. In embodiments, the beam 182 is formed from metal and extends through a mold cavity of the mold assembly (not shown) such that the first jaw 112 of the ligation clips 100 are over molded about and supported on the metal beam 182 after the ligation clips 100 are removed from the mold assembly (not shown). The beam 182 eliminates the need to handle the small ligation clips individually during loading of a multi-fire clip applicator (not shown).

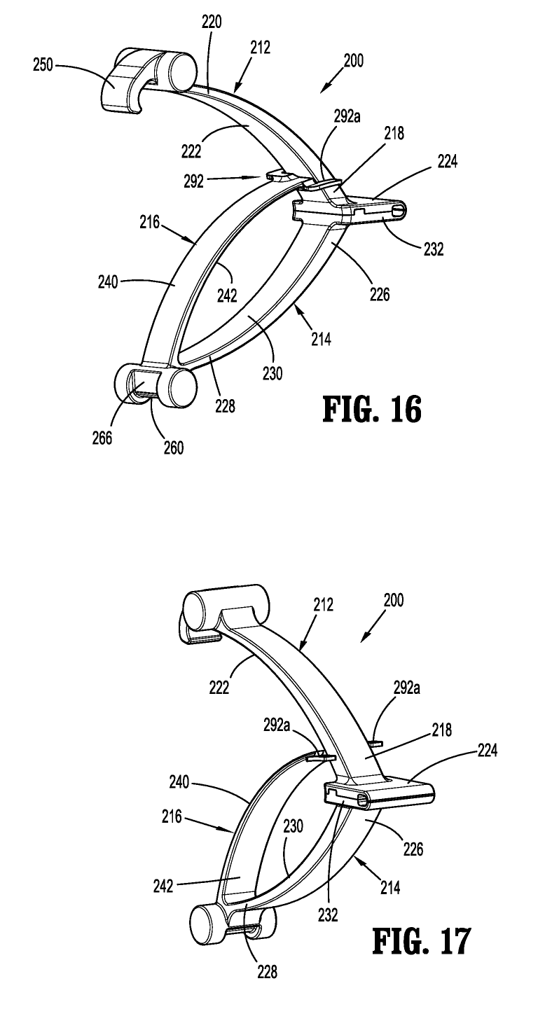

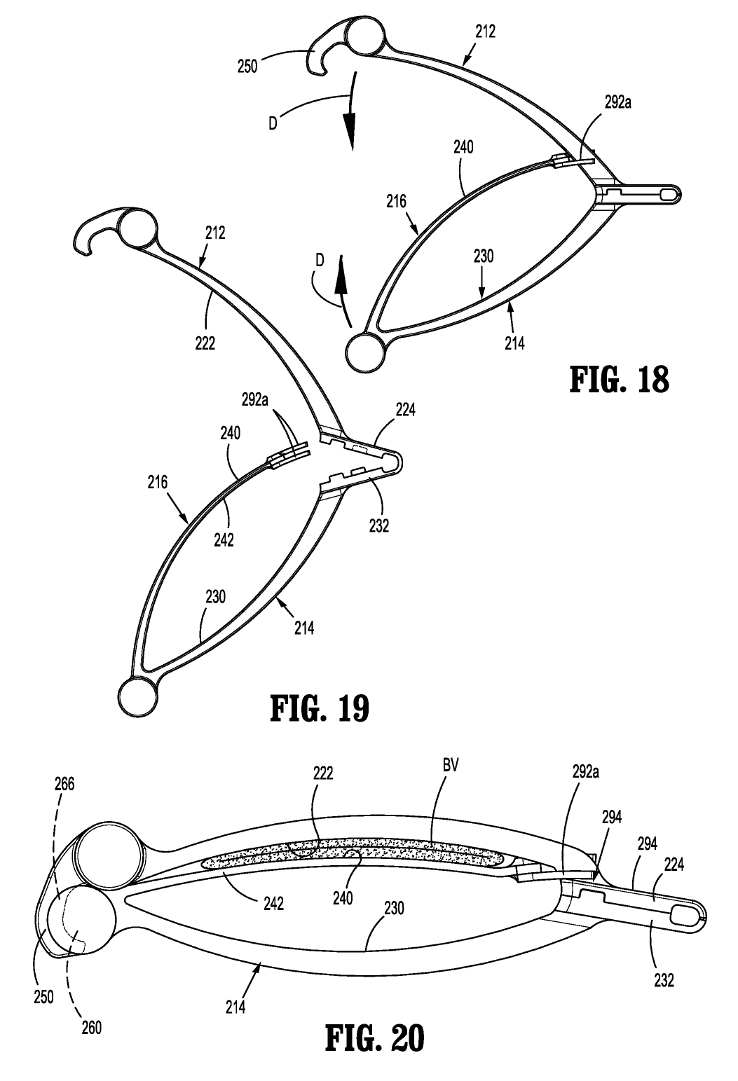

FIGS. 16-20 illustrate another exemplary embodiment of the presently disclosed ligation clip which is shown generally as ligation clip 200. Referring to FIGS. 16-18, the ligation clip 200 is substantially similar to the ligation clips 10 and 100. Accordingly, only the differences between the ligation clip 200 and the ligation clips 10 and 100 will be described in detail herein. The ligation clip 200 includes a first jaw 212, a second jaw 214, and a central beam 216. The first jaw 212 includes a proximal portion 218, a distal portion 220, and a clamping surface 222. In embodiments, the proximal portion 218 of the first jaw 212 includes a base portion 224. The second jaw 214 includes a proximal portion 226, a distal portion 228, and an inner surface 230. In embodiments, the proximal portion 226 of the second jaw 214 includes a base portion 232 that is engaged with the base portion 224 of the first jaw 212 to secure the clamping surface 222 of the first jaw 212 in opposition to the inner surface 230 of the second jaw 214.

The central beam 216 of the ligation clip 200 extends from the distal portion 228 of the second jaw 214 towards the proximal portion 218 of the first jaw 212 and engages the clamping surface 222 of the first jaw 112. The central beam 216 has a proximal end that includes a forked structure 292 including spaced tines 292a. The forked structure 292 is positioned to engage the first jaw 212 such that one of the spaced tines 292a is positioned on each side of the first jaw 212. As described above in regard to central beams 16, 116, the central beam 216 may be curved and include a first side 240 defining a convexity that faces the clamping surface 222 of the first jaw 212 and a second side 242 that defines a concavity that faces the inner surface 230 of the second jaw 214. In embodiments, the central beam 216 is integrally and/or monolithically formed with the second jaw 214. Alternately, the central beam 216 can be formed separately from the second jaw 214 and resiliently mounted to the second jaw 214 using a pivot member (not shown) and a biasing member (not shown).

Referring to FIGS. 19 and 20, the ligation clip 200 is applied to a body vessel “BV” in the same manner that the ligation clips 10, 100 are applied to a body vessel “BV”. More specifically, when the ligation clip 200 is used to clamp a body vessel “BV” (FIG. 20), the ligation clip 200 is placed in the open position about the body vessel “BV” with the body vessel “BV” positioned between the first convex side 240 of the central beam 216 and the clamping surface 222 of the first jaw 212. In the open position, the flexible beam 216 is spaced from both the clamping surface 222 of the first jaw 212 and the inner surface 230 of the second jaw 214. The first and second jaws 212, 214 are clamped about the body vessel “BV” by moving the first and second jaws 212, 214 in the direction indicated by the arrows “D” in FIG. 19. The central beam 216 is resilient and applies a controlled pressure to the body vessel “BV” to allow proper tissue sealing to minimize the likelihood of ischemic tissue damage. As the first and second jaws 212, 214 continue to be moved in the direction of arrows “D” towards the clamped position (FIG. 20), the first locking element 250 moves along the cam surface 266 (FIG. 16) and eventually passes off of the cam surface 266 such that the first locking element 250 snaps into engagement with the second locking element 260 to retain the first and second jaws 212, 214, respectively, in the clamped position. As the first and second jaws 212, 214 are moved towards the clamped position (FIG. 20), the tines 292a of the forked structure 292 move along opposite sides of the first jaw 212 and engage an outer surface 294 of the base portion 224. The forked structure 292 maintains the central beam 216 in alignment with the first jaw 212. Engagement between the tines 292a and the outer surface 294 of the base portion 224 of the first jaw 212 prevents movement of the forked structure 292 in relation to the first jaw 212 to increase the force applied to the body vessel “BV” as the ligation clip 200 is moved to the fully clamped position (FIG. 20). Once again, the resilience of the central beam 216 applies a controlled pressure to the body vessel “BV” to seal the body vessel “BV” between the central beam 216 and the clamping surface 222 of the first jaw 212.

The ligation clip 200 can be released from the body vessel “BV” in the same manner that the ligation clips 10, 100 are released from the body vessel “BV”.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A ligation clip comprising:
a first jaw having a proximal portion, a distal portion, and a clamping surface;
a second jaw having a proximal portion, a distal portion, and an inner surface that faces the clamping surface of the first jaw, the proximal portion of the first jaw being connected to the proximal portion of the second jaw, wherein the first jaw is pivotable in relation to the second jaw between open and clamped positions; and
a central beam positioned between the clamping surface of the first jaw and the inner surface of the second jaw, the central beam formed of a resilient material and being spaced from the clamping surface of the first jaw and the inner surface of the second jaw in the open position of the first and the second jaws.

2. The ligation clip of claim 1, wherein the central beam extends from the distal portion of the second jaw towards the clamping surface of the first jaw.

3. The ligation clip of claim 2, wherein the central beam is integrally formed with the second jaw.

4. The ligation clip of claim 2, wherein the central beam has a proximal end that is positioned adjacent to the clamping surface of the first jaw when the ligation clip is in the open position.

5. The ligation clip of claim 2, wherein the central beam is dimensioned to engage the clamping surface of the first jaw such that a proximal end of the central beam is deformed downwardly towards the inner surface of the second jaw.

6. The ligation clip of claim 5, wherein the proximal end of the central beam includes a forked structure, the forked structure including first and second spaced tines and the first jaw including opposite sides, wherein each of the first and second tines is positioned on one of the opposite sides of the first jaw.

7. The ligation clip of claim 6, wherein the proximal portion of each of the first and second jaws includes a base portion, the spaced tines being positioned to engage the base portion as the first and second jaws are moved towards the clamped position.

8. The ligation clip of claim 7, wherein the base portion is rectangular.

9. The ligation clip of claim 2, wherein the central beam has a length and a thickness, the thickness of the central beam decreasing along at least a portion of the length from a distal end of the central beam to a proximal end of the central beam.

10. The ligation clip of claim 1, wherein the first and second jaws have a length and a thickness, the thickness of the first and second jaws decreasing along at least a portion of the length of the first and second jaws from a proximal end of the first and second jaws to a distal end of the first and second jaws.

11. The ligation clip of claim 1, wherein the ligation clip is formed from a biocompatible polymeric material that is selected from the group consisting of acetal polyoxymethylene (POM), polyethylene terephthalate (PET), polybutylene terephthalate (PBT), polyoxymethylene, polyetheretherketone (PEEK), polypropylene, and polyethylene.

12. The ligation clip of claim 1, wherein the central beam is positioned and configured to be spaced from the first and second jaws in the clamped position.

13. The ligation clip of claim 1, wherein each of the first and second jaws includes a pair of bosses, each of the pair of bosses being configured to engage an applicator to apply the ligation clip to tissue.

14. The surgical ligation clip of claim 13, wherein each of the pair of bosses projects outwardly beyond an outer surface of one of the first and second jaws.

15. The ligation clip of claim 1, wherein the first jaw includes a first locking element and the second jaw member includes a second locking element, the first locking element being movable into engagement with the second locking element to retain the first and second jaws in the clamped position.

16. The ligation clip of claim 1, wherein a proximal end of the base portion of the first jaw is secured to the proximal end of the base portion of the second jaw by a living hinge.

17. The ligation clip of claim 16, wherein the base portion of the first and second jaws are retained in juxtaposed relation to each other by interlocking structure.

18. The ligation clip of claim 17, wherein the interlocking structure includes protrusions formed on one of the first and second jaws and recesses defined in the other of the first and second jaws.

19. The ligation clip of claim 1, wherein the distal portion of the first jaw includes a first locking element and the distal portion of the second jaw includes a second locking element, the first locking element being configured to engage with the second locking element to retain the first and second jaws in the clamped position.
